(19) United States
(12) Patent Application Publication
(10) Pub. No.: US 2007/0163575 A1
(43) Pub. Date: Jul. 19, 2007

(54) Nebulizer

(76) Inventor: Antonio M. Rojas JR., Miami Shores, FL (US)

Correspondence Address: ROBERT C. KAIN, JR., 750 SOUTHEAST THIRD AVENUE, SUITE 100, FT LAUDERDALE, FL, 333161153, US

(21) Appl. No.: 11/324,090

(22) Filed: Dec. 30, 2005

(51) Int. Cl. A61M 11/00

(52) U.S. Cl. 128/200.21; 128/200.14

(57) ABSTRACT

The nebulizer includes a housing, a compressed gas inlet, an orifice ejecting the gas as a gas jet and liquid medicament entrained into the gas jet and forming a spray. The spray is directed to a spheroidal diffuser (preferably a ball) mounted beneath a cap forming a substantially cylindrical space within which swirls or spins the deflected spray jet. An opposing diffuser plate, opposite the downwardly open end of the cap, provides additional deflection as does specially shaped legs spacing the cap form the deflector-diffuser plate. The diffuser plate has a deflection edge about its periphery (either a full deflector edge, or several 45 degree arcuate edges, or small arcuate segments) which edge causes additional deflection and diffusion of the aerosol.

The present invention relates to a nebulizer for converting a medication solute into an aerosol for inhalation delivery of medicine or nutritional supplements and a method of nebulizing such solute.

### BACKGROUND OF THE INVENTION

Nebulizers have been used to create an aerosol for medication for many years. As used herein, the term “medication” refers to any liquid that enhances a respiratory condition of a person or mammal or that treats a medical condition or other respiratory condition of the person or mammal or a solute that is recommended to be delivered into a respiratory system of a person or mammal. Several prior art patent disclosures are discussed below that relate to nebulizers.

In the disclosure for U.S. Pat. No. 6,338,443 to Piper, FIG. 8 illustrates upwards flow towards a convex diffuser or aerosol amplifier 44 (col. 5, line 28, line 55, line 65, col. 6, lines 1-10). Aerosol particles 58 rise above the flat deflector plate which plate includes a spray hole. Col. 6, line 54. The particles also flow off a chamfer on the flat plate deflector. A containment bottle (FIG. 7, Piper '443), with a downwardly open lip and upward aerosol holes, is twice as radially large as the flat plate deflector. U.S. Pat. No. 4,333,450 to Lester, in FIG. 2, shows a needle pin 8 within a downwardly open cup. The cup is twice the radial size as the opposing deflector plate. Additionally, the deflector plate has a concave nozzle face. U.S. Pat. No. 5,503,139 to McMahon, FIG. 4 discloses a concave diffuser 108 above a flat diffuser plate. Aerosol particles rise vertically in the McMahon system.

Other prior art disclosures include U.S. Pat. No. 5,875,774 which shows another nebulizer. U.S. Pat. No. 5,738,086 to McMahon, FIG. 1 discloses convex diffuser. Col. 4, line 21. Fluid flow is upward. U.S. Pat. No. 5,579,757 to McMahon shows FIG. 1 same as McMahon '086. U.S. Pat. No. 5,506,100 to Surzycki, FIGS. 7A, 7B discloses convex diffuser 17 (FIG. 7A) and various recycle passages. See FIGS. 7B, 7C. See also FIG. 16. U.S. Pat. No. 5,458,136 to Jaser, FIG. 6 discloses an assembly for producing aerosol pulses. U.S. Pat. No. 5,235,969 to Bellm, FIG. 2 and FIG. 3 discloses a convex diffuser 41 supported by legs 49. Legs 49 define spaces beneath diffuser 41. U.S. Pat. No. 4,792,097 to Kremer, Jr., FIG. 3 does not appear to have diffuser bulb. U.S. Pat. No. 4,566,452 shows another nebulizer. U.S. Pat. No. 4,588,129 to Shanks, FIG. 4 discloses diffuser 20 (FIG. 4). U.S. Pat. No. 4,512,341 to Lester, FIG. 1 shows diffuser bulb 27. FIGS. 3, 4. U.S. Pat. No. 4,456,179 to Kremer, FIG. 3 does not seem to show diffuser bulb. U.S. Pat. No. 4,301,970 to Craighero, FIG. 1 shows, in the lower section of FIG. 1, a description of the operation of impeller 28 (which rotates) and deflectors 90 is found at col. 4, lines 26-42. Downward flow is noted therein. U.S. Pat. No. 4,101,611 to Williams, FIGS. 2, 7 do not show a diffuser bulb. U.S. Pat. No. 3,864,326 to Babington, FIGS. 1, 6 shows various bulbs. U.S. Pat. No. 3,842,833 to Ogle, FIG. 2 shows a bulb 62 in the jet stream. U.S. Pat. No. 3,838,686 to Szekely, FIGS. 1, 2 shows a convex structure 22 within a concave deflection cup 23. FIGS. 1, 2. U.S. Pat. No. 3,762,409 to Lester, FIGS. 1, 5 shows convex diffuser 62 (col. 5, line 10) within a concave shield. Col. 3, line 30. U.S. Pat. No. 3,744,722 to Burns, FIGS. 1-2 generally shows a bulb diffuser. U.S. Pat. No. 3,630,196 to Bird, FIGS. 2, 4, 5 show a bulb diffuser. U.S. Pat. No. 3,584,621 to Bird, FIG. 2 shows a bulb diffuser. U.S. Pat. No. 3,525,476 to Boling, FIG. 5 shows a bulb diffuser. U.S. Pat. No. 3,522,806 to Szekely, FIG. 1 shows a bulb diffuser. U.S. Pat. No. 3,353,536 to Bird, FIGS. 2, 5, 6 shows a bulb diffuser. U.S. Pat. No. 3,301,255 to Thompson, FIG. 5 shows a bulb diffuser 57. U.S. Pat. No. 3,172,406 to Bird, FIGS. 3, 4 generally shows a ball diffuser. U.S. Pat. No. 3,069,097 to Cheney, FIG. 6 generally shows a ball diffuser. U.S. Pat. No. 3,018,971 to Cheney, FIGS. 1, 2 generally shows a ball diffuser. U.S. Pat. No. 2,840,417 to Dorsak, FIG. 3 shows a ball diffuser at the end of a round tube end. See also, U.S. Pat. No. 2,726,896 to McKinnon, FIGS. 1, 4. U.S. Pat. No. 2,605,764 to Adams, FIG. 3 generally shows a ball diffuser. U.S. Pat. No. 2,274,669 to Curry, FIG. 2 generally shows aball diffuser. U.S. Pat. No. 1,150,238 to Winbray, the sole figure shows a convex diffuser. U.S. Pat. No. RE33,642 to Lester, FIG. 1 shows a convex diffuser. U.S. Pat. No. RE30,046 to Amerongen, FIG. 2 generally shows a ball diffuser.

### OBJECTS OF THE INVENTION

It is an object of the present invention to provide a nebulizer which converts a solute medication into an aerosol for delivery by inhalation.

It is another object of the present invention to provide a method for nebulizing a medication solute.

It is a further object of the present invention to provide a nebulizer which employs legs which stabilize the spinning spray which is deflected off a spheroidal diffuser, which may be a ball diffuser.

It is a further object of the present invention to provide legs which stabilize the spinning spray of medication with small arcuate curved surfaces.

It is another object of the present invention to provide a nebulizer which is moderately quiet during operation.

It is a further object of the present invention to provide a nebulizer with a deflection edge about a periphery of a diffuser plate which reduces the accumulation of droplets at the bottom region of the nebulization chamber.

It is another object of the present invention to employ legs or posts which raise the cap above the diffusion plate and which have a radial span which causes the spinning fluid under the cap to further aerosolize. The legs have a squared off lower, radially facing edge which cuts the 360 degree jet spray and has a sloped region above the radially facing edge which adds to the spinning of the jet fluid and further aerosolization of the fluid.

Another object of the present invention is to minimize the wasted medicament to be nebulized.

### SUMMARY OF THE INVENTION

The nebulizer converts a solute into an aerosol for inhalation delivery of solute medication. The nebulizer includes a housing, a compressed gas inlet which is in communication with a source a compressed gas which is, in turn, coupled to the nebulizer. An orifice in the nebulizer permits the passage of compressed gas therethrough to form a gas jet. A supply of liquid containing the solute medication is entrained into the gas jet, typically by capillary action, to form a spray. The spray is directed to a spheroidal diffuser having a spheroidal surface upon which the spray is caused to impinge and thereby form an aerosol. The spheroidal diffuser may be a ball or an irregular sphere. A cap forms a substantially cylindrical space about and above the spheroidal diffuser. The cap's interior surface further deflects the aerosol. An opposing diffuser plate, opposite the downwardly open end of the cap, provides additional deflection of the aerosol. The spray passes through the diffuser plate via a secondary orifice. The downwardly open edge of the cap generally falls in the same plane as defined by the diffuser plate. The diffuser plate has a deflection edge about its periphery (either a full, 360 deflector edge, or a pair of 45 degree arcuate edge regions or small arcuate segmental regions) which edge causes additional deflection and diffusion of the aerosol. A plurality of legs space the cap apart from the diffuser plate. The legs themselves provide supplemental deflection of the aerosol.

### BRIEF DESCRIPTION OF THE DRAWINGS

Further objects and advantages of the present invention can be found in the detailed description of the preferred embodiments when taken in conjunction with the accompanying drawings in which:

FIG. 1 diagrammatically illustrates a nebulizer chamber;

FIG. 2 diagrammatically illustrates a nebulizer in accordance with the principles of the present invention;

FIG. 3 diagrammatically illustrates a nebulizer with a flat plate diffuser and a peripheral ledge;

FIG. 4A diagrammatically illustrates a nebulizer with a flat plate diffuser and a small arcuate cutout acting as a deflection edge segment (multiple cutouts or arcuate deflection edge segments may be defined along the periphery of the deflector plate);

FIGS. 4B, C and D diagrammatically illustrate flow patterns in the space beneath the cap;

FIG. 5 diagrammatically illustrates a curved top cap diffuser;

FIG. 6 diagrammatically illustrates supporting the spherical diffuser via a depending wall mount; and

FIG. 7 diagrammatically illustrates a cap with an inner curved wall segment and wedged shaped legs.

### DETAILED DESCRIPTION OF THE PREFERRED EMBODIMENTS

The present invention relates to a nebulizer and a method of nebulizing solute medication into an aerosol. Similar numerals designate similar items throughout all the drawings.

FIG. 1 shows nebulizer canister 10 having a compressed gas inlet 12, a solute jar 14, and an inhaler cap 16, and an aerosol outlet 16.

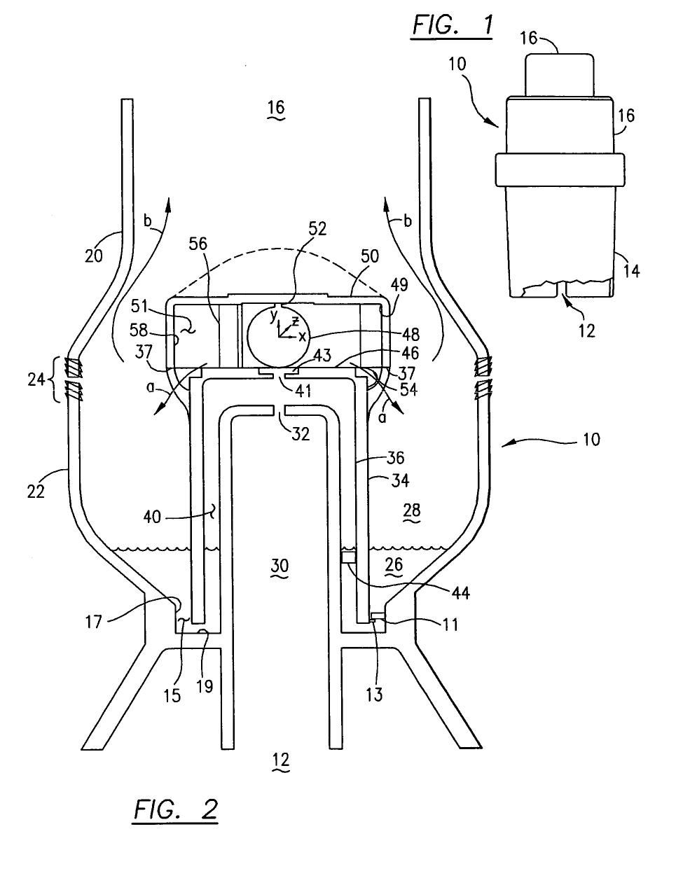

FIG. 2 diagrammatically shows many more details of nebulizer 10. Nebulizer 10 includes upper body 20 which is, in the illustrated embodiment, threadably attached to lower body element 22 by threads 24 which are diagrammatically illustrated in FIG. 2. Other attachment mechanisms (snap on, tongue and groove, twist and channel release) may be used. A solute medication 26 is retained in lower space 28 of bottom element body 22. Beginning with compressed gas inlet 12, a gas inlet column 30 leads to primary orifice 32. An intermediate structure 34 is removably fixed about interior tube 36 which interior tube defines the gas inlet column 30. Structure 34 is removably mounted on or in nebulizer 10 by an attachment system 11, on nebulizer 10—wall 22 and by complimentary attachment system 13 on structure 34. The attachment mechanism may be a snap-on, a tongue and groove, a twist-screw or other removable attachment system.

To decrease waste medicament 26, the lower region of nebulizer 10 defines an open top annular ring area 15 defined by vertical wall segment 17 and base wall segment 19. The open top annular ring captures small amounts of nebulizer liquid and facilities the suctioning of medicament into annular upwards channel 40 as explained later. Prior art nebulizers have a flat bottom, with no open top annular ring 15, or simply have a slopped bottom. Excess liquid medicament collects the bottom regions of prior art devices. The annular ring in the present invention has a volume to retain about 0.2-0.3 cc of fluid.

When compressed gas passes through primary orifice 32, a jet is created which pulls the solute up passage 40 between intermediate structure 34 and inner tube 36. Principally, the solute 26 is pulled by capillary action and other pneumatic and hydrodynamic forces due to the gas jet ejected from primary orifice 32. A secondary orifice 41 is immediately above primary orifice 32. These orifices are coaxial. One or more deflectors 44 (arcuately positioned) are mounted intermediate to space apart tubular body 34 and inner tubular body 36. These deflectors also channel the upwardly directed medicament liquid to air ejection port 32.

The jet from primary orifice 32 causes solute to be entrained into the gas jet exiting orifice 32 thereby forming a spray which enters secondary orifice 41. The output of secondary orifice 41 is a larger open space 43 in a generally flat diffuser plate 46. Open space 43 is an annular open ring atop the diffuser plate 46. The spray carrying entrained solute and leaving secondary orifice 41 strikes and impinges upon a spheroidal diffuser 48. Preferably, spheroidal diffuser 48 is a sphere which causes the spray to diffuse in substantially 360 degrees in substantially three dimensions, that is, in the x (horizontal), y (vertical) and z (normal to the plane of the drawing) directions. Of course, spheroidal diffuser 48 is mounted to cap 50 by a support element or beam 52. Therefore, diffuser ball or spheroid 48 cannot cause diffusion of the spray completely opposite secondary orifice 41. Preferably, spheroid 48 is a ball with a common diameter.

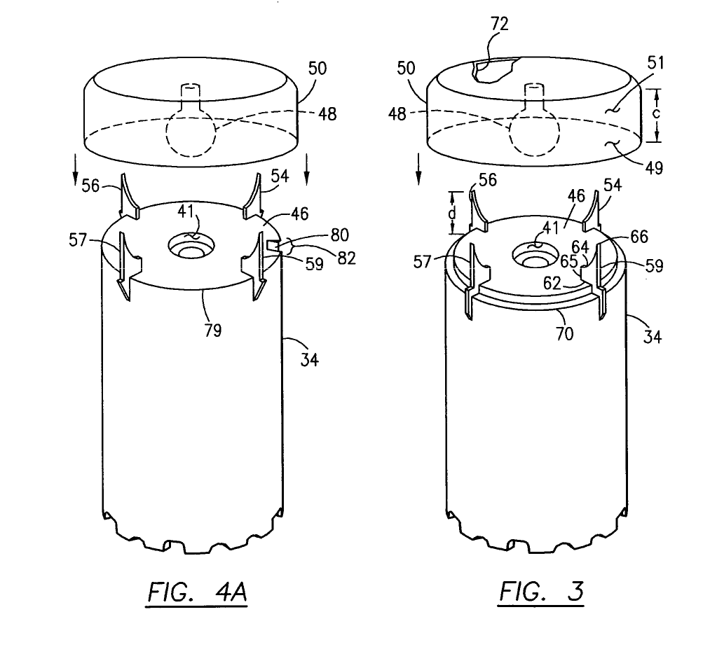

Cap 50 is mounted to intermediate cylinder 34 with a plurality of legs, two of them being illustrated as legs 54 and 56 in FIG. 2. Four legs are preferred. A better view of these legs are shown in FIG. 3. In FIG. 2, three legs are utilized but only legs 54, 56 are illustrated. The legs are disposed at equidistant peripheral positions about diffuser plate 46. Spray leaving secondary orifice 41 strikes diffuser ball 48 and after striking ball 48 the resulting aerosol—fluid is deflected in a plurality of directions including laterally (plus x and minus x) such that the initially diffused aerosol becomes further aerosolized and strikes interior wall 58 of cap 50. Effectively, cap 50 defines a substantially cylindrical space 51 about and above diffuser ball 48 as well as above spray ejecting orifice 41. The downward edge of the cap 50, that is, edge 37, falls in substantially the same plane as the top surface of deflector or diffuser plate 46. The aerosol spray swirls about in cap space 51 and increased aerosolization of the medication has been observed.

Legs 54, 56 slice or control the diffusion and turbulence of the spray after the spray leaves ball surface 48 thereby enhancing aerosolization of the spray. The spray leaves cylindrical chamber 51 as shown by downwardly directed arrows “a” and then the aerosolized solute travels upward as shown by arrows “b” to leave aerosol outlet 16.

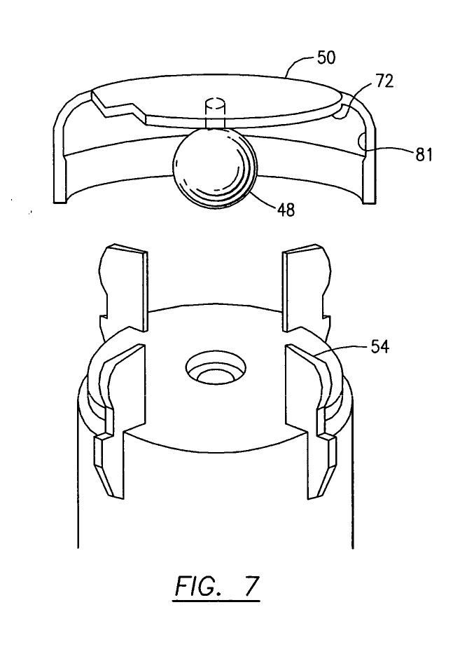

FIG. 3 shows that deflector or diffuser flat plate 46 includes, about its periphery, a deflection edge 70 which extends about its entire periphery. It is important to note that the plane of flat diffuser plate 46 is substantially coextensive with the downward edge 37 of cap 50 and cap opening 49 of cylindrical region 51. Further, in FIG. 3, four legs 54, 56, 57, 59 retain cap 50 above flat diffuser plate 46. These legs have a narrow or thin arcuate aspect (in the arcuate dimension relative to orifice 41) as these legs face secondary orifice 41. FIG. 7 shows legs 54 as a wedge with the thin end of the wedge pointed towards the centerline of the ejection jet port. In FIG. 3, the base 62 of the these legs, see base 62 of leg 59, is radially wide as compared to a radially narrow top 66. The radially wide base 62 has a squared off edge 65 which faces the ejection port and causes additional nebulization of the ejected fluid. An intermediate curve or curvaceous portion 64 of leg 59 is presented to the spray leaving diffuser ball 48. The small arcuate thickness or aspect of legs 54, 56, 57, 59 assists in segmenting the deflected spray from diffuser ball 48. Further, the curvaceous slopped face 64 of each of the legs 54, 56, 57, 59 also assists in further causing the spray to spin beneath cap 50 and aerosolize, thereby creating additional aerosol. The peripheral ledge 70 about the entire periphery of diffuser plate 46 seems to decompress the aerosol solute in cylindrical space 51 and reduce noise. Ledge 70 reduces the noise generated by the nebulizer. In one embodiment, plate 46 is 8-9 mm in diameter and the cap 50 has an open diameter of 14 mm. Posts rather than legs may be used but the radial span 62 of the base, the squared off presenting edge 65 and slopped curved edge segment 64 seem to improve nebulization of the medicament.

In a preferred embodiment, the vertically span “c” of cap 50 and the diameter of cylindrical space 51 defines an approximate volume of 0.7 cc. The diameter of diffuser ball 48 is approximately 4 mm. The height of the legs “d” is substantially equivalent to the cylindrical height “c” of cylindrical chamber 51. In a further improved embodiment, the interior upper corner 49 (FIG. 1) of the cap 50 is curved which further enhances spinning and multi-dimensional flow of ejected medicament. The legs, upon visual observation of the nebulization chamber, seem to stabilize the spinning liquid within cylindrical chamber 51, especially when the nebulizer is at an angle off the vertical axis which angulation is typical during normal operation. The curve 64 on the legs seems to enhance fluid spinning in chamber 51 and return non-aerosolized spray back into chamber 51 thereby increasing aerosolization. Further, the sharp presenting arcuate edges 65 of the legs 54, 56, 57, 59 also create additional turbulence and enhance aerosolization. The utilization of a deflection edge, such as edge 70 about the deflector plate 46, seems to reduce the noise of the nebulizer.

Experiments have shown that when chamber 51 is too small, nebulizer noise is very high. In addition, there is a visible bubbling of liquid all over inside the cap and the aerosol output of the system is very small. Further, the output opening has many bursting tiny bubbles that splash medicament out to the inner walls of the nebulizer 10. However, when the chamber is too big, although the noise decreases, the amount of aerosolized solute decreases and this reduces the efficiency of the nebulizer. Tests were conducted comparing the present design with other commercially available nebulizers. The present nebulizer delivered 3 cc of standardized medicine in 11-12 minutes. The manufacturer of the medicine had prescribed that the delivery should occur within 6-15 minutes. Other nebulizers utilizing this same standardized medicine delivered 3 cc of medicine within 10-12 minutes. Tests further showed that the best delivery time of 3 cc of standardized medicine for competitive designs was 11 minutes but excessive amounts of un-nebulized liquid (about 0.6-1.0 cc) remained in the competitor's systems. Nebulizers of the present invention leave 0.3-0.4 cc of liquid remaining, thereby indicating a higher degree of nebulization and delivery into the patient. These experiments utilized a diffuser ball of 4 mm diameter and a cap 50 having an interior diameter of 14 mm. Experiments further show that when the interior diameter of cap 50 was reduced to 10 mm, the noise of the nebulizer was too high and the output on an aerosolized basis based upon the amount of standardized fluid was too low. When the diameter cap was increased to 16 mm, the nebulizer noise was reduced but the time of delivery for delivering 3 cc of standardized medicine was too long. It is believed that this dimensional range (within 10-16 mm) is important based upon standard compressed gas input and the degree of aerosolization of liquid.

Further experiments have shown that a curves on the inner edge of cap inner surface 72 create further turbulence and spinning of fluid in cylindrical chamber 51 that decrease noise and increase aerosolization of the solute. See FIG. 3.

The utilization of a deflection edge or ledge 70 (other deflection edges are discussed later) significantly prevents unwanted tiny droplets from flowing out of the generally cylindrical chamber 51 of the nebulizer. The use of a deflection edge is thought to enable some type of decompression of the air pressure in chamber 51. With a deflection edge about the entire periphery of deflection plate 46 (FIG. 3), visual observations noted no accumulation of droplets along the bottom of the chamber 51. Further, the noise produced by the nebulizer was reduced. Without deflection edges, poorer performance was noted. The aerosol output with a deflection edge was viewed to be stronger, of higher density and better quality than experiments of a flat deflected plate 46 without such deflective edges. The deflective edge may be a pair of opposing 45 arcuate degree segments.

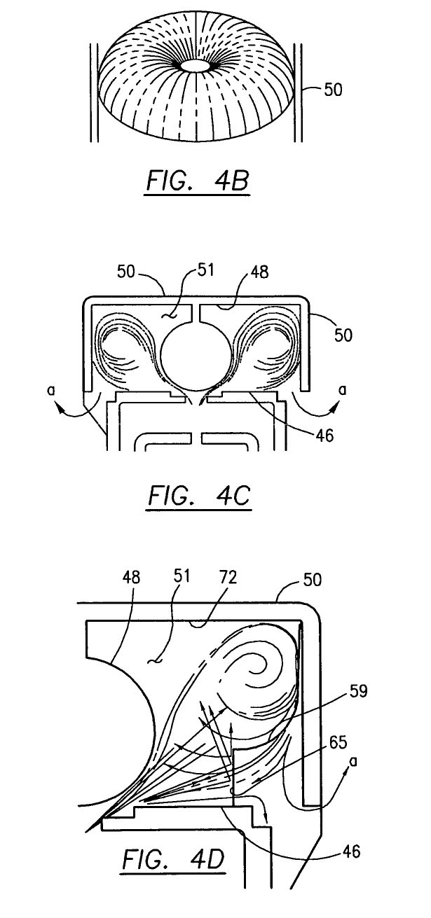

FIG. 4A shows a deflection edge 80 having a partial arcuate span 82. A plurality of arcuate cutouts 80 may be defined about periphery 79 of flat deflective plate 46. Although the utilization of a plurality of small arcuate deflection cutouts 80 improved the performance of nebulizer (compared with a flat plate with no deflectors), a plurality of arcuate deflectors or an entire peripheral ledge 70 (FIG. 3) provided the best nebulizer.

FIG.4B shows the 360 degree spinning spray developed by the present system. FIG. 4C shows a cross-sectional view of the spinning spray wherein the spray leaving ball 48 moves upward (y+) and then moves downward (y−) and then strikes plate 46 which causes the spray to again to move upward (y+) thereby causing a spin. The spin continues until the fluid is light enough to be ejected in direction a. FIG. 4D shows the splitting of the flow by leg 59, the deflection by presenting edge 65 and the multi-dimensional flow beneath cap 50.

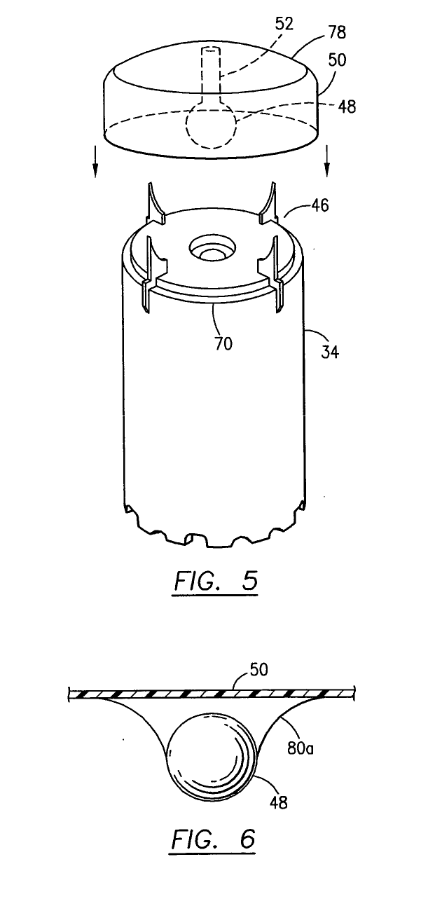

FIG. 5 shows cap 50 with a round or curved top 78 rather than a flat top 72 of FIG. 3.

FIG. 6 shows that diffuser ball 48 may be mounted or supported on cap 50 by a depending wall 80 rather than a depending member or bar 52.

FIG. 7 shows a wedge shaped legs 54 and a curved, interior corner 81 of cap 50. The curved corner increases spinning and aerosolization. The outer shape of a domed top also reduces the accumulation of previously aerosolized medicament.

It is thought that nebulization and aerosolization of this medication solute is not created simply by turbulence or atomization but by collision or progressively breaking the bubbles with a spinning or swirling fluid beneath the cap and the vertical legs 54, 56 which cause additional aerosolization. This enhances spinning or swirling of fluid and maintains or enhances the velocity of the fluid in the interior of the cap. This increases aerosolization. Visual observation of several models of the current nebulizer resulted in observations that the spray leaving the secondary orifice created a spinning of liquid in cylindrical chamber 51 and this spinning produced more compressed bubbles. The increasing bubbles resulted in greater degrees of aerosolization and delivery of the solute medication.

The claims appended hereto are meant to cover modifications and changes within the scope and spirit of the present invention.

What is claimed is:

1. A nebulizer for conversion of a solute into an aerosol for inhalation delivery of solute medication comprising:
a nebulizer housing;
a compressed gas inlet in communication with a source of compressed gas coupled to said nebulizer;
an orifice through which said compressed gas passes from the gas inlet to form a gas jet;
a supply of liquid containing solute medication which is entrained into said gas jet to form a spray;
a spheroidal diffuser having a spheroidal surface upon which said spray is caused to impinge to form an aerosol;
a cap forming a substantially cylindrical space both about and above said spheroidal diffuser, said cap having an interior surface further deflecting said aerosol;
a diffuser plate through which passes said spray, said spheroidal diffuser and said cap disposed above said diffuser plate such that additional deflection of said aerosol occurs thereon;
said diffuser plate having a deflection edge about its periphery which is substantially co-extensive with respect to said cap and said cylindrical space; and,
a plurality of legs spacing said diffuser plate away from said cap, said legs providing supplemental deflection of said aerosol.

2. A nebulizer as claimed in claim 1 wherein said spheroidal diffuser and spheroidal surface and diffuser plate diffuses said spray and said aerosol 360 degrees in substantially three dimensions.

3. A nebulizer as claimed in claim 1 wherein said diffuser plate is substantially planar and is substantially co-extensive with respect to the open end of said cylindrical space defined by said cap.

4. A nebulizer as claimed in claim 3 wherein said deflection edge of said diffuser plate is a substantially peripheral chamfer about said periphery of said diffuser plate.

5. A nebulizer as claimed in claim 3 wherein said deflection edge of said diffuser plate is defined by a plurality of angulated cut-outs about said periphery of said diffuser plate.

6. A nebulizer as claimed in claim 3 wherein said deflection edge of said diffuser plate is a peripheral ledge about said periphery of said diffuser plate.

7. A nebulizer as claimed in claim 1 wherein said legs have a thin arcuate aspect, relative to said spheroidal diffuser, and have a radially wide base mounted to said diffuser plate and have a radially narrow top mounted to said cap.

8. A nebulizer as claimed in claim 7 wherein said legs have a curvaceous edge intermediate said radially wide base and said radially narrow top, said curvaceous edge presented to said aerosol formed by said spheroidal diffuser.

9. A nebulizer as claimed in claim 1 wherein said cap has a dome top encasing an upper end of said substantially cylindrical space.

10. A nebulizer as claimed in claim 1 wherein said cap has a flat top encasing an upper end of said substantially cylindrical space.

11. A nebulizer as claimed in claim 1 wherein said diffuser plate defines an annular ring about a secondary orifice for said spray.

12. A nebulizer as claimed in claim 2 wherein said diffuser plate is substantially planar and substantially co-extensive with respect to an open end defined by said cylindrical space under said cap.

13. A nebulizer as claimed in claim 12 wherein said deflection edge of said diffuser plate is one of a substantially peripheral chamfer about said periphery of said diffuser plate; a plurality of angulated cut-outs about said periphery of said diffuser plate; aperipheral ledge about said periphery of said diffuser plate, or a plurality of arcuate ledge segments.

14. A nebulizer as claimed in claim 13 wherein said legs have a thin arcuate aspect, relative to said spheroidal diffuser, a radially wide base mounted to said diffuser plate and a radially narrow top mounted to said cap and an intermediate curvaceous edge presented to said aerosol formed by said spheroidal diffuser.

15. A nebulizer as claimed in claim 14 wherein said cap has one of a dome top or a flat top encasing an upper end of said substantially cylindrical space.

16. A nebulizer as claimed in claim 15 wherein said diffuser plate defines a secondary orifice for said spray.

17. A nebulizer for conversion of a solute into an aerosol for inhalation delivery of solute medication comprising:
a nebulizer housing;
a compressed gas inlet in communication with a source of compressed gas coupled to said nebulizer;
an orifice through which said compressed gas passes from the gas inlet to form a gas jet;
a supply of liquid containing solute medication which is entrained into said gas jet to form a spray;
a spheroidal diffuser having a spheroidal surface upon which said spray is caused to impinge to form an aerosol;
a cap defining a substantially cylindrical space about and above said spheroidal diffuser further deflecting said aerosol;
a diffuser plate below said spheroidal diffuser and said substantially cylindrical space additionally deflecting said aerosol;
a deflection edge about a periphery of said diffuser plate substantially co-extensive with said cylindrical space; and,
a plurality of legs mounting said cap above said diffuser plate.

18. A nebulizer as claimed in claim 17 wherein said spheroidal diffuser diffuses spray and aerosol 360 degrees in substantially three dimensions.

19. A nebulizer as claimed in claim 18 wherein said deflection edge is one of a substantially peripheral chamfer about said periphery of said diffuser plate, a plurality of angulated cut-outs about said periphery of said diffuser plate, a peripheral ledge about said periphery of said diffuser plate or a plurality of arcuate ledge segments.

20. A nebulizer as claimed in claim 17 wherein said legs have a narrow arcuate dimension relative to said spheroidal diffuser, have a radially wide base mounted to said diffuser plate and a radially narrow top mounted to said cap with an intermediate curvaceous edge.

21. A nebulizer as claimed in claim 17 wherein said cap has one of a dome top or a flat top at an upper end of said substantially cylindrical space.

22. A nebulizer as claimed in claim 17 wherein said nebulized housing has, in its interior region, a lower annular open top ring for accumulation of medication.

23. A method for nebulizing a solute into an aerosol for inhalation delivery of solute medication comprising:
feeding compressed gas into an inlet in a nebulizer;
forming a gas jet via an orifice through which passes said compressed gas;
forming a spray by entraining a supply of liquid containing solute medication into said gas jet;
diffusing said spray in substantially 360 degrees in substantially three dimensions by impinging said spray on a spheroidal diffuser and forming an aerosol thereat;
further deflecting said aerosol in a defined cylindrical space both about and above said spheroidal diffuser;
additionally deflecting said spray with a diffuser plate disposed blow said cylindrical space;
causing edge deflection of said spray at the periphery of said diffuser plate; and
supplementally deflecting said spray with a plurality of legs peripherally spaced about said diffuser.

24. A method for nebulizing as claimed in claim 23 including causing edge deflection of said spray over substantially all of the periphery of said diffuser plate.

25. A method for nebulizing as claimed in claim 24 including causing edge deflection of said spray over a plurality of discreet arcuate portions of the periphery of said diffuser plate.
